(19) United States
(12) Patent Application Publication
(10) Pub. No.: US 2025/0204757 A1
(43) Pub. Date: Jun. 26, 2025

(54) HANDPIECE FOR A FLEXIBLE ENDOSCOPE OR A FLEXIBLE ENDOSCOPIC INSTRUMENT AND FLEXIBLE ENDOSCOPE OR FLEXIBLE ENDOSCOPIC INSTRUMENT

(71) Applicant: Karl Storz SE & Co. KG, Tuttlingen (DE)

(72) Inventors: Katharina ENDRESS, Tuttlingen (DE); Tobias LOTTES, Tuttlingen (DE); Daniel ULMSCHNEIDER, Tuttlingen (DE)

(73) Assignee: Karl Storz SE & Co. KG, Tuttlingen (DE)

(21) Appl. No.: 18/849,886

(22) PCT Filed: Mar. 22, 2023
(86) PCT No.: PCT/EP2023/057333

§ 371 Date: Sep. 23, 2024

(30) Foreign Application Priority Data
Mar. 25, 2022 (DE) 10 2022 107 123.5

(51) Int. Cl. A61B 1/005

(52) U.S. Cl. CPC A61B 1/0052; A61B 1/0057

(57) ABSTRACT

A handpiece for a flexible endoscope or a flexible endoscopic instrument having a bendable shaft portion includes a housing, a support frame, a control element, and an actuating element, and a Bowden cable with a cable element for bending the bendable shaft portion. The handpiece has a guide device having a guide element with guide recesses for passing the cable element and a clamping device with at least one clamping element. The clamping device is detachably arranged on the distal side of the guide element and is non-positively connected to the support frame by means of the at least one clamping element, such that the guide element is held in position between the clamping device and the distal end of the support frame and the cable element can be guided through the at least two opposite guide recesses in a proximal direction to the control element.

### CROSS-REFERENCE TO RELATED APPLICATIONS

This application is the U.S. national stage of PCT/EP2023/057333 filed on Mar. 22, 2023, which claims priority of German Patent Application No. DE 10 2022 107123.5 filed on Mar. 25, 2022, the contents of which are incorporated herein.

### TECHNICAL FIELD

The invention relates to a handpiece for a flexible endoscope or a flexible endoscopic instrument having a bendable shaft portion, wherein the handpiece has a housing with a distal end and a proximal end, a support frame, a control element, and an actuating element, wherein the support frame is connected to the housing on the inside, at least one Bowden cable having a cable element for bending the bendable shaft portion of the flexible endoscope or the flexible endoscopic instrument can be assigned to the handpiece, the control element for actuating the cable element of the at least one Bowden cable is held by means of the support frame, and the control element can be actuated by means of the actuating element, wherein the handpiece has a guide device for guiding the at least one Bowden cable and/or the cable element. Furthermore, the invention relates to a flexible endoscope or a flexible endoscopic instrument having a shaft and a bendable shaft portion.

### BACKGROUND

Bendable instruments and endoscopes which can be operated manually and/or robotically are often used in medical and non-medical applications. Reusable endoscopes or instruments often use a plurality of steering wires, which are arranged around pivoting elements of a mechanical joint system on the distal side and fastened to a drivable swash plate on the proximal side. For endoscopes and instruments that are only used once, such a complex design and assembly is not feasible. For disposable endoscopes and instruments, it is known to use a circumferential Bowden cable or a few Bowden cables for the joint mechanics on the distal and proximal sides.

US 2020/0229684 A1 discloses a handpiece for an endoscope, wherein the Bowden cable sheaths are glued with adhesive into through-holes of an anchor block, wherein the anchor block is arranged within the handpiece. However, the gluing process required for this purpose requires more time for assembly due to the curing time of the adhesive. In addition, if the adhesives are not fully cured and/or crosslinked, they may subsequently release undesirable substances, and, if the Bowden cable sheath is in an unfavorable position, there is a risk that the pull wire will become glued to the Bowden cable sheath. Above all, the through-holes through the anchor block restrict the guidance of the Bowden cable and the pull wire in terms of their spatial orientation.

### SUMMARY

The object of the invention is to improve upon the prior art.

The object is achieved by a handpiece for a flexible endoscope or a flexible endoscopic instrument having a bendable shaft portion, wherein the handpiece has a housing having a distal end and a proximal end, a support frame, a control element, and an actuating element, wherein the support frame is connected to the housing on the inside, at least one Bowden cable having a cable element for bending the bendable shaft portion of the flexible endoscope or the flexible endoscopic instrument can be assigned to the handpiece, the control element for actuating the cable element of the at least one Bowden cable is held by means of the support frame, and the control element can be actuated by means of the actuating element, wherein the handpiece has a guide device for guiding the at least one Bowden cable and/or the cable element, and the guide device has a guide element, having at least two opposite guide recesses for passing the cable element through, and a clamping device having at least one clamping element, wherein the guide element is detachably arranged at a distal end of the support frame, the clamping device is detachably arranged on the distal side of the guide element and is non-positively connected to the support frame by means of the at least one clamping element, such that the guide element is held in position between the clamping device and the distal end of the support frame, and the cable element can be guided through the at least two opposite guide recesses in a proximal direction to the control element.

Thus, a handpiece is provided that has simple and cost-effective guidance of the cable element and/or the Bowden cable by means of the guide device. It is particularly advantageous for the guide element having the at least two opposite guide recesses to be spatially fixed to the support frame in a non-positive manner by means of the clamping device and for an integral bond, such as welding or gluing, not to be required. By clamping the guide element using the clamping device, these two components can be easily and quickly assembled on the support frame as a guide device. They can also be disassembled quickly, and the individual guide element and the individual clamping device can be disposed of or recycled separately. This means that the handpiece is designed for single use—for example, for a disposable endoscope or a disposable instrument.

Above all, the handpiece can be quickly and easily adapted to different flexible endoscopes or instruments, e.g., having different shaft diameters, since, depending upon the shaft diameter in each case, one or more Bowden cables with correspondingly adapted cross-sections are usually used. In order to adapt to the properties of each endoscope or instrument and/or the Bowden cables, only the guide element and/or the clamping device must be replaced. Thus, it is possible to easily adapt to the requirements of each endoscope or instrument by using different guide elements, e.g., having guide recesses of different sizes and/or made at different heights of the guide element and/or by using different clamping devices—for example, with different-shaped clamping elements and/or different clamping forces. Thus, with the exception of the detachable and replaceable guide element and/or the detachable and replaceable clamping device, the handpiece can always have the same construction for different endoscopes and/or instruments.

An essential concept of the invention is based upon the fact that an identical handpiece is provided for various flexible endoscopes and instruments, in which the guide device for guiding the at least one Bowden cable and/or the cable element is connected to the support frame in a non-positive manner by means of the at least one clamping element and thus without an integral bond, such that simple assembly and fabrication of the handpiece is possible by means of different guide elements, clamping devices, and/or clamping elements in order to quickly and easily adapt the guide device to the requirements of the endoscope or instrument in each case. Due to this simple usability and assembly of the guide element and the clamping device, the spatial course of the at least one Bowden cable and/or the cable element coming from the distal-side mechanical joint system can also in a targeted manner be spatially aligned by means of the guide device and can be guided towards the control element.

The following terminology is explained:

A ‘handpiece’ is in particular a handheld part and/or retaining part for an endoscope or instrument. In particular, the handpiece can be a handle for manual and/or automatic operation. In particular, the handpiece has a housing. For example, the housing can be made up of two lateral handle shells that are connected to one another. At the distal end of the handpiece, the handpiece can be connected, e.g., by means of an adapter, to the proximal end of a shaft of the endoscope or instrument.

An ‘endoscope’ is in particular a medical or industrial appliance for endoscopic examination and inspection of a human or animal body cavity and/or an industrial cavity, such as a tube. The endoscope has in particular a handpiece, a shaft, a light source, a light guide, and a camera. The endoscope is in particular a video endoscope, which has digital image recording and image transmission. A video endoscope is in particular a chip-on-the-tip (COTT) endoscope, wherein the image sensor is designed as a chip, e.g., a CMOS chip, and is arranged on the distal end portion of the shaft of the video endoscope. The image data recorded by the image sensor can in particular be transmitted electronically through the shaft in the proximal direction to the handpiece and further to a display system and/or to an image processing unit in order to display the endoscopic image to the user. In addition to human and veterinary applications, an endoscope and/or video endoscope can, however, also be used for industrial purposes—for example, for visual inspection in hard-to-reach cavities. In industrial applications, an endoscope is often referred to as a borescope.

An ‘endoscopic instrument’ is in particular any mechanical or mechanical-electrical operating unit which can be used in particular for manipulating human or animal tissue. The endoscopic instrument has in particular a handpiece, a flexible shaft, and a tool and/or an optical system for observing a viewing area. A flexible endoscopic instrument usually does not have its own optics for taking an endoscopic image, but can be used in particular together with a flexible endoscope. The endoscopic instrument may in particular have a gripping tool, a cutting tool, a needle holder, a clip placement device, and/or another type of tool.

A ‘flexible endoscope’ or a ‘flexible endoscopic element’ is understood in particular to mean that the endoscope or the endoscopic instrument has a flexible shaft. A ‘flexible shaft’ is in particular a partially or fully flexible elongated tube. The flexible shaft is in particular designed to be inserted into a cavity to be examined endoscopically—for example, a body cavity or a technical opening in industrial applications. Typically, the shaft has an outer diameter in a range of 4 mm to 10 mm. The flexible shaft has, in particular, at least one operating channel on the inside for flushing or for passing operating instruments through. The flexible shaft has at least one bendable shaft portion. The ‘bendable shaft portion’ refers in particular to a bendable distal end portion of the shaft. In order to bend the shaft portion, the flexible shaft has, in particular on the inside, a distal-side mechanical joint system with joint members arranged side-by-side in a row in the longitudinal direction, and at least one circumferential Bowden cable guided around the outside of the joint members on both sides. The housing of the Bowden cable can be fastened, on the distal side, in front of the first joint member or, in the distal end portion, in front of the camera head—for example, by gluing.

‘Distal side’ and ‘distal’ are understood to mean an arrangement and/or a corresponding end or portion that is close to the body and therefore remote from the user. Accordingly, ‘proximal side’ and ‘proximal’ are understood to mean an arrangement and a corresponding end or portion close to the user and thus remote from the body. Thus, the ‘proximal direction’ is a direction towards the proximal end of the handpiece and/or the endoscope or endoscopic instrument.

A ‘support frame’ (also called a ‘bearing block’) is in particular a supporting structure inside the housing. The support frame is connected in particular to the housing and/or a handle shell on the inside. The support frame is in particular designed having two opposite support arms, between which the control element is arranged. The control element is connected to the two support arms of the support frame, in particular by means of a shaft.

A ‘control element’ is in particular an element which causes the cable element of the at least one Bowden cable to move, and thus the bendable shaft portion to bend. A control element can, for example, be an idler pulley or a steering wheel.

An ‘actuating element’ is in particular an element whose movement actuates the control element. An actuating element can, for example, be an articulation lever with a handle at the end, wherein the lever itself is guided through the housing of the handpiece and connected to the control element.

A ‘Bowden cable’ is in particular a movable machine element for transmitting a mechanical movement. A Bowden cable has in particular a housing (also called an outer cable) that is flexible but pressure-resistant in the pulling direction, and a cable element arranged inside the housing (also called an inner cable, pull cable, or pull wire). The cable element is movable within the housing, in particular in the longitudinal direction of the Bowden cable. The cable element is in particular a steel wire or wire cable. By moving the cable element by means of the control element, the cable element is displaced relative to the housing of the Bowden cable in the longitudinal direction of the Bowden cable, whereby the bendable shaft portion can be bent by a set amount in a set direction by means of the distal mechanical joint system. The housing of the Bowden cable is, at least in part, substantially cylindrical. The Bowden cable can, for example, have an outer diameter in the range of 0.10 mm to 3.00 mm, in particular 0.30 mm to 1.00 mm, preferably 0.40 mm to 0.75 mm.

A ‘guide device’ is in particular a device which guides the at least one Bowden cable and/or the cable element in the handpiece in the proximal direction to the control element. The guide device has in particular a guide element, having at least two opposite guide recesses, and a clamping device having at least one clamping element.

A ‘guide element’ can in particular be any component which has guide recesses on two opposite sides for passing the cable element through. A guide element can in particular be a three-dimensional rectangular component. Preferably, the guide element is a plate having a small wall thickness. The guide element has in particular a metal and/or an alloy, such as stainless steel.

A ‘guide recess’ is in particular an aperture through the dimension of the guide element in the proximal direction. A guide recess can be a hole, a groove, or a slot. In the cross-section transverse to the proximal direction, a guide recess is preferably formed directly in the left and right side walls of the guide element in each case. For example, a guide opening can be made there, which passes deeper into the interior of the guide element transversely to the proximal direction in the form of a slot and is continuous in the proximal direction.

A ‘clamping device’ is in particular a three-dimensional component which can be clamped to the support frame by means of the at least one clamping element, and can thus be detachably connected to the support frame in a non-positive connection. When the guide device is assembled, the guide element is clamped in particular surface-to-surface between the proximal side of the clamping device and the distal side of the support frame, and is thus spatially held in place. In this case, the guide element is preferably aligned such that its two opposite larger surfaces lie surface-to-surface and flush against the proximal side of the clamping device and the distal side of the support frame. The clamping device has in particular plastic, such as polypropylene.

A ‘clamping element’ is in particular an elastic element which is or can be connected directly to the support frame in a non-positive manner. A clamping element can, for example, be a latching hook having a latching lug or a spring clip.

A ‘non-positive connection’ is in particular a connection in which a normal force is applied to the surfaces to be interconnected. This prevents the surfaces from moving relative to one another due to static friction. In the non-positive connection, the retaining force of the clamping element is used to fix the clamping device and the guide element to the support frame by automatically locking thereto. The non-positive connection is in particular a clamping connection. The non-positive connection is in particular reversible and releasable, in particular by loosening the clamping element.

An ‘integral bond’ is in particular a bond in which the bonding partners are held together by atomic or molecular forces. An integral bond is in particular a non-releasable bond which can be separated only by destroying the connecting means. An integral bond is, for example, welding or gluing.

In a further embodiment, the handpiece has the at least one Bowden cable, a second Bowden cable, and/or further Bowden cables, each having a cable element.

Thus, instead of a circumferential Bowden cable, two or more Bowden cables can also be guided inside the handpiece by means of the guide device.

In the case of two Bowden cables, two cable elements working against one another can be guided on opposite sides of the control element. These two cable elements can also be connected to one another on the proximal side and thus be designed as a continuous cable element.

A ‘second Bowden cable’ and ‘further Bowden cables’ correspond in their design and function to the Bowden cable defined above, but they can be routed differently and at a distance from one another in the handpiece and/or through the flexible shaft to the bendable shaft portion.

In order to ensure a stable and secure non-positive connection between the at least one clamping element and the support frame, the support frame has at least one recess, an undercut, and/or at least one projection element in which and/or around which the at least one clamping element engages.

Thus, a defined point or region of the support frame is provided at which the at least one clamping element or elements can apply their clamping force by engaging in and/or around said support frame.

A ‘recess’ is in particular an incision and/or an opening. The recess may, for example, be a notch, a groove, and/or another type of incision in the surface of the support frame.

An ‘undercut’ is in particular a construction element which protrudes freely from the support frame.

A ‘projection element’ is understood to mean in particular an element which projects beyond the surface of the support frame. The projection element can in particular widen conically and/or in a stepped fashion in the direction of the clamping region. Preferably, the projection element has one or more corners for one or more clamping elements to engage around.

In a further embodiment, the support frame has, on both sides, an aperture in the proximal direction, which corresponds to the corresponding guide recess, for passing the cable element or the cable elements through, or has a smaller width than the guide element.

As a result, the guide element or the guide elements coming from the two opposite guide recesses in the guide element can be guided on both sides in the proximal direction to the control element either freely or guided by the apertures on both sides of the support frame.

A ‘corresponding aperture’ is understood to mean a right-hand and left-hand aperture through a distal wall of the support frame, which is aligned with the guide element. In this case, the apertures in the distal wall of the support frame are arranged in particular in such a way that the cable elements guided by the guide recesses in the guide element in the proximal direction or the portions of the cable element on both sides can be guided through the apertures in the support frame. Preferably, the apertures in the support frame have a larger free opening diameter than the guide recesses, and therefore the guide element or the guide elements guided on both sides can be spatially guided flexibly and thus so as to be laterally and/or vertically displaceable relative to the control element. This also makes it possible to implement different diameters of the control element.

An ‘aperture’ is in particular a continuous opening through the distal wall of the support frame. An aperture can be a hole in the surface of the distal wall or a recess in the right and left sides of the distal wall of the support frame in the proximal direction. Thus, the wall thickness of the distal side wall can have a groove or a notch on both sides from the outside. Preferably, the aperture is at least partially round or circular such that friction caused by the cable element passing inside the aperture is avoided.

In principle, however, the support frame can also have a smaller width than the guide element such that the cable element or the cable elements passing through the guide recesses in the proximal direction is/are guided freely around the outside of the distal wall of the support frame towards the control element.

In order to clamp around a projection element on two opposite sides and/or to at least partially engage therearound on both sides, the clamping device has at least two opposite, interacting latching hooks, each having a latching lug, which at least partially engage around a first projection element on an upper side of the support frame.

Thus, a clip closure is provided by the two opposite interacting latching lugs and the latching hooks.

A ‘latching lug’ is an element that protrudes with respect to the latching hook. A latching lug can be designed in particular as a pin, projection, and/or offset. The latching lug is arranged in particular at the free end of a latching hook. The latching hook is in particular elastically deformable, such that the two latching hooks can be pushed around the projection element on both sides before the latching lugs, for example, each engage around a corner of the projection element on both sides.

In a further embodiment, the clamping device has two further opposite interacting latching hooks, each having a latching lug, which at least partially engage around a second projection element on an underside of the support frame.

Thus, the clamping device can be connected in a non-positive manner by means of one or two pairs of latching hooks having latching lugs on the upper side and/or underside of the support frame. With the non-positive connection on the underside and the upper side of the support frame, the clamping device thus clamps around the support frame at its distal end largely from the outside.

In order to also use the clamping device for guiding the at least one Bowden cable or Bowden cables, the clamping device on both sides has at least one lateral guide projection, having a continuous groove in the proximal direction for guiding the at least one Bowden cable or Bowden cables to the guide recesses in the guide element.

Consequently, by means of the guide projections on both sides, the Bowden cable or Bowden cables coming from the shaft can in a targeted manner be directed towards the guide recesses in the guide element.

A ‘guide projection’ is understood to mean a part of the clamping device which protrudes laterally and thus transversely to the proximal direction, and which has a continuous groove in the proximal direction. Thus, each lateral guide projection can be designed, for example, in the form of a protruding wing. This limits the width of the clamping device transversely to the proximal direction, and the clamping device does not have to be solid in this transverse direction, thus saving upon weight and space in the handpiece.

In a further embodiment, the clamping device has two lateral guide projections on both sides, each with a continuous groove in the proximal direction, wherein the two guide projections are arranged one after the other in the proximal direction on both sides, and/or the corresponding two grooves are arranged in opposite directions to one another.

By means of the two guide projections arranged one after the other on one side, guidance of the Bowden cable in the proximal direction can in a targeted manner be spatially adjusted. Since the two consecutive grooves are arranged in opposite directions to one another—for example, the groove of the distal-side guide projection is open at the top, and the groove of the subsequent proximal-side guide projection is open at the bottom—a through-region for the Bowden cable or Bowden cables is defined in the cross-section. This allows Bowden cables with different diameters and/or with tolerance deviations to be guided through this opening region, and the arrangement of the two consecutive grooves can be used to align in a targeted manner the Bowden cable in the proximal direction.

In order to guide the portions of the Bowden cable or Bowden cables on both sides and thus the corresponding cable elements at a greater lateral distance from one another in the proximal direction, the lateral guide projection arranged on the proximal side has a larger lateral dimension than the lateral guide projection arranged on the distal side, such that the at least one Bowden cable or Bowden cables expands or expand laterally outwards in the proximal direction on both sides through the two consecutive grooves.

Thus, the Bowden cable or Bowden cables can be guided in a targeted manner to the control element by the two guide projections spaced apart from one another in the proximal direction, both in terms of their orientation in the proximal direction and transversely to the proximal direction, and thus at a predetermined lateral distance. This makes it easier to route the Bowden cable or Bowden cables to the outside and also allows their alignments to be extended in a targeted manner in a V-shape to the control element. Consequently, an extension of the distance in the proximal direction can already be realized with the Bowden cable on both sides.

In a further embodiment of the handpiece, a proximal end of a housing of the at least one Bowden cable or of the Bowden cable rests on a distal side of the guide element around the guide recess, and/or is integrally bonded.

Thus, the proximal end of the housing of the Bowden cable abuts the distal side of the guide element around the corresponding guide recess in a defined manner, and only the cable element is guided further through the guide recess in the proximal direction to the control element. This allows the cable element to move in the longitudinal direction and thus the axial direction of the Bowden cable within the housing, and thus allows the bendable shaft portion of an endoscope or endoscopic instrument to bend. The end face of the proximal end of the housing can be integrally bonded, e.g., glued, to the distal side of the guide element. Additionally or alternatively, the housing can also be glued into the groove in a guide projection of the clamping device.

In order to easily introduce the cable element or the cable elements laterally from the outside into the opposing guide recesses, the at least two opposite guide recesses each have a guide opening, wherein the guide openings are arranged on both sides in opposite side walls of the guide element in a cross-section that is transverse to the proximal direction.

Since the guide openings are made laterally in the side walls from the outside and do not pass therethrough within the distal surface, the cable element and/or cable elements can be easily threaded in from the outside.

In a further embodiment, the at least two opposite guide recesses have, proceeding from each guide opening, a constant recess height, a stepped recess height, and/or a tapering recess height along a recess width.

This allows cable elements having different diameters to be guided through each of the guide recesses in the proximal direction in a stable position, without any lateral displacement.

‘Recess width’ refers in particular to the dimension of the recess in a direction transverse to the proximal direction. A ‘recess height’ is understood to be a dimension of the recess in the vertical direction and thus transverse both to the recess width and to the proximal direction. Proceeding from the guide openings, which are on both sides, of the opposite guide recesses, the guide recesses narrow in cross-section towards the center of the guide element.

In order to provide a high clamping force and a large clamping surface by means of the clamping device, the guide element is designed as a guide plate, wherein a wall thickness of the guide plate is arranged in the proximal direction.

Thus, the opposite plate surfaces lie surface-to-surface against the clamping device on the distal side and surface-to-surface against the distal side of the support frame.

A ‘guide plate’ is in particular a flat element with a uniform thickness (wall thickness) throughout, which is delimited on two opposite sides by a flat surface that has a large extent in relation to its thickness.

In a further embodiment, the guide plate has an upper recess and a lower recess in a cross-section transverse to the proximal direction, and the support frame has an upper retaining projection corresponding to the upper recess and a lower retaining projection corresponding to the lower recess.

This allows the guide plate to be easily placed on the distal side of the support device before the guide plate is clamped to the support frame by means of the clamping device. Due to the upper and lower retaining projections protruding from the distal side of the support frame, the guide plate can be placed on the distal side of the support frame without slipping downwards. Of course, the support frame can also have only a lower retaining projection on its distal side, and the guide plate can have only a lower recess. The upper and lower recesses in the guide plate can each have any shape—for example, it can be a triangular or a rectangular recess in the surface of the guide plate. Each retaining projection of the support frame accordingly has the correspondingly opposite design.

In a further aspect of the invention, the object is achieved by a flexible endoscope or a flexible endoscopic instrument having a shaft and a bendable shaft portion, wherein the bendable shaft portion can be bent by means of at least one Bowden cable, and having a handpiece arranged at a proximal end of the shaft, wherein the handpiece is a handpiece as described above.

Thus, a flexible endoscope or a flexible endoscopic instrument having a handpiece that is inexpensive to manufacture and easy to assemble is provided for single use, such that re-use and the otherwise necessary autoclaving can be dispensed with. Above all, a largely identical handpiece, in which only the guide element and the clamping device can have different designs, provides an easy-to-use endoscope or endoscopic instrument, especially for the user, which does not require the user to adjust to the change between different types of endoscopes or instruments when operating the handpiece.

The invention will be explained in more detail below with reference to an exemplary embodiment.

### BRIEF DESCRIPTION OF DRAWINGS

FIG. 1 is a three-dimensional view of an endoscope having a handpiece and a flexible shaft,

FIG. 2 is a three-dimensional view of the open handpiece,

FIG. 3 is a three-dimensional view of a guide device when not assembled and a bearing block of the handpiece, and

FIG. 4 shows the guide device assembled on the bearing block.

### DETAILED DESCRIPTION OF PREFERRED EMBODIMENTS

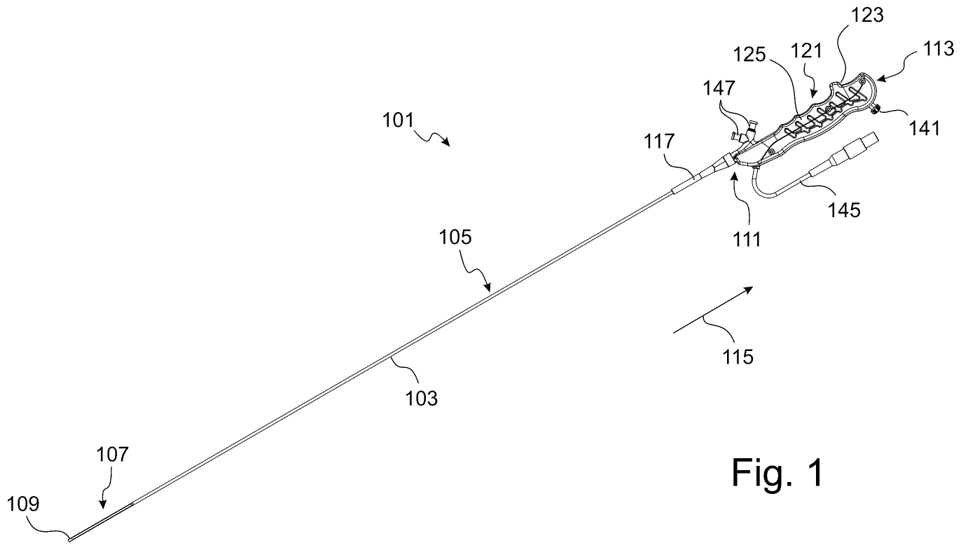

An endoscope 101 has a shaft 103 having a flexible shaft portion 105 and a distal bendable shaft portion 107. A camera head 109 is arranged in a distal end portion of the bendable shaft portion 107. A proximal end of the shaft 103 is connected to a distal end 111 of a handpiece 121 by means of an adapter 117. The handpiece 121 has a housing 123, which is formed by a left-hand and a right-hand handle shell 125. In a proximal portion in front of a proximal end 113 of the handpiece 121, the handpiece 121 has an articulation lever 141 which is guided inwards into the housing 123. In a distal portion in front of the distal end 111, the handpiece 121 has two valve bodies 147 and a power cable 145 (FIG. 1).

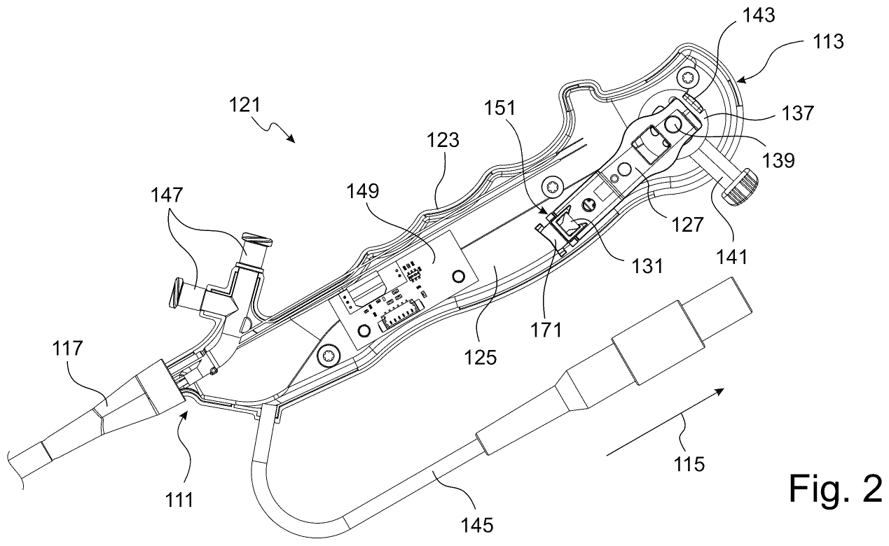

In the interior, the handpiece 121 (FIG. 2 shows a view into the interior of the handpiece 121 with the left handle shell 125 removed) has a circuit board 149 and a bearing block 127 on the proximal side. The bearing block 127 has an upper bearing arm 128 and a lower bearing arm 130, which are connected to a distal wall 132 on the distal side. A drive shaft 139 is guided through the proximal end of the upper bearing arm 128 and the lower bearing arm 130 such that the bearing block 127 carries the drive shaft 139, to which a steering wheel 137 is fastened. On the proximal side, the control wheel 137 is secured by a safety clip 143. The articulation lever 141 is connected to the steering wheel 137.

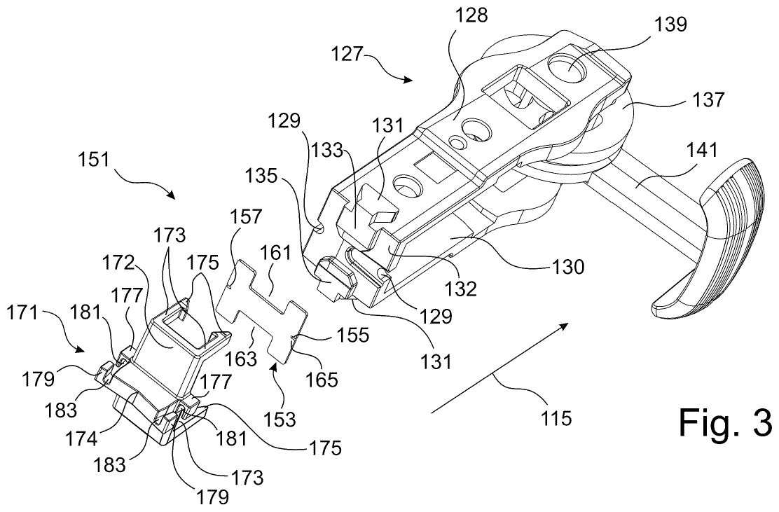

In an assembled state, a guide device 151 having a guide clip 171 and a guide plate 153 for a Bowden cable 191 having a housing 193 and a pull cable 195 is arranged on the bearing block 127 on the distal side (see FIGS. 2, 3, and 4). A clip projection 131 is arranged on the upper side of the upper bearing arm 128. The clip projection 131 on the upper side of the upper bearing arm 128 merges into an upper retaining projection 133 on the distal wall 132 of the bearing block 127. Furthermore, the lower bearing arm 130 also has on its underside a clip projection 131, which merges into a lower retaining projection 135 on the distal wall 132. In the distal wall 132, on the right side and the left side each an aperture opening 129 is made in a side wall of the distal wall 132, which openings are continuous in a proximal direction 115 (see FIG. 3).

The guide plate 153 has a right guide slot 155, a left guide slot 157, and an abutment side 165. Furthermore, the guide plate 153 has an upper recess 161 which corresponds to the upper retaining projection 133 of the bearing block 127, and a lower recess 163 which corresponds to the lower retaining projection 135 of the bearing block 127. Thus, the guide plate 153 has an H-shape in cross-section. The guide clip 171 has a vertical wall 172 and a base 174. Two latching hooks 173 with inward-facing latching lugs 175 are arranged at an angle of 90° on both sides of the upper side of the vertical wall 172. Likewise, two latching hooks 173, which are not fully visible in FIG. 3, with inward-facing latching lugs 175, are arranged on the underside of the guide clip 171, opposite those arranged on the upper side. The guide clip 171 has, on either side of the base 174, a distal guide wing 179 with a distal groove 183 that is open at the top and, following therefrom at a distance in the proximal direction 115, has a proximal guide wing 177 on either side having a proximal groove 181 that is open at the bottom. The proximal and distal grooves 181 and 183 are continuous in the proximal direction 115.

To assemble the guide plate 153 and the guide clip 171 on the bearing block 127, the following work steps are carried out:

The guide plate 153 is placed on the distal wall 132 of the bearing block 127 so that the upper retaining projection 133 and the lower retaining projection 135 of the bearing block 127 each protrude into the upper recess 161 and the lower recess 163, thereby holding the guide plate 153 on the distal wall 132. The guide clip 171 is then pushed in the proximal direction 115 onto the retained guide plate 153 and the distal end of the bearing block 127. The two opposite upper latching hooks 173 and the lower latching hooks 173, which are not fully visible in FIG. 3, laterally engage around each of the upper clip projection 131 and the lower clip projection 131. Due to the resilient, elastic design of the latching hooks 173 and the clip projection 131 that widens conically in the proximal direction 115, the two opposite latching hooks 173 are widened when pushed on until their latching lugs 175 engage around the proximal-side corners on either side of each clip projection 131 and are locked in place. Due to this non-positive connection, the guide plate 153 is clamped and spatially fixed between the inner side of the vertical wall 172 of the guide clip 171 and the distal wall 132 of the bearing block 127.

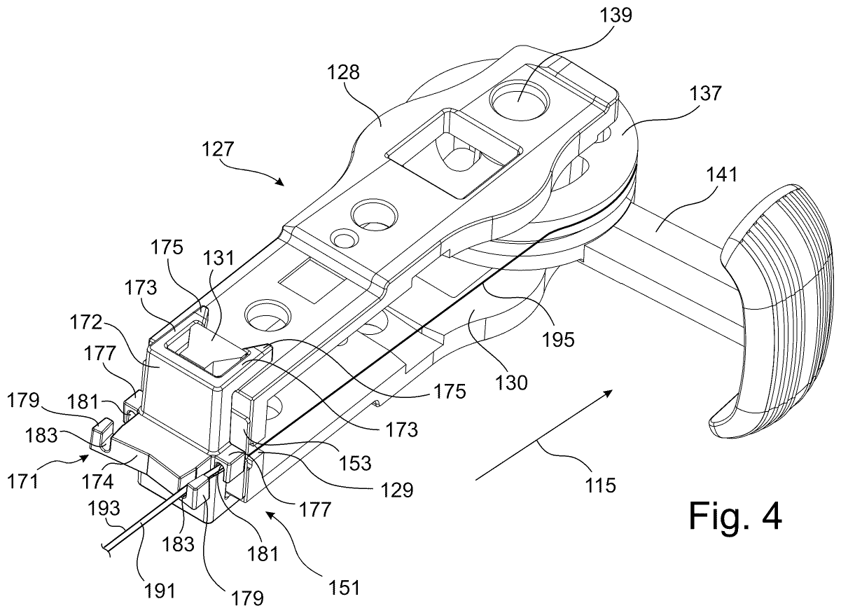

The Bowden cable 193 is now threaded through the distal groove 183 of the distal guide wing 179, which groove is open at the top, and the proximal groove 181 of the proximal guide wing 179, which groove is open at the bottom and follows in the proximal direction 115, wherein the housing 193 thereof abuts, by means of its proximal end, the abutment side 165 of the guide plate 153 around the right guide slot 155, where it is glued. Alternatively, the proximal end of the housing 193 is integrally bonded to the guide plate 153 before being clamped by means of the guide clip 171. The inner pull cable 195 is guided through the right guide slot 155 of the guide plate 153 and through the subsequent aperture opening 129 in the distal wall 132 of the bearing block 127 and guided back around the outside of the control wheel 137 counter to the proximal direction 115. In FIG. 4, the Bowden cable 193 and the pull cable 195 are shown guided only on the right side of the guide device 151 and the bearing block 127, wherein, on the left side, the Bowden cable 193 and the pull cable 195 are guided in a similar way to the right side.

A user actuates the articulation lever 141 to bend the bendable shaft portion 107 of the endoscope 101. Due to the arrangement of the steering wheel 137 on the rotatable drive shaft 139 for conjoint rotation therewith, which drive shaft is connected to the articulation lever 141, the steering wheel 137 is rotated by pivoting the articulation lever 141, and the pull cable 195 is displaced within the housing 193 of the Bowden cable 191. As a result, the bending of the bendable shaft portion 107 is manually controlled by the user of the endoscope 101.

Thus, a handpiece 121 having a guide device 151 is provided, in which the guide clip 171 and the guide plate 152 of the guide device 151 can be reversibly fastened to the distal wall 132 of the bearing block 127 in a detachable and non-positive manner, thus optimally guiding the Bowden cable 191 and the pull cable 195 by laterally extending the cable element guidance in the proximal direction 115 to the control wheel 137.

1. Handpiece for a flexible endoscope or a flexible endoscopic instrument having a bendable shaft portion, wherein the handpiece has a housing with a distal end and a proximal end, a support frame, a control element, and an actuating element, wherein the support frame is connected to the housing on the inside, at least one Bowden cable having a cable element for bending the bendable shaft portion of the flexible endoscope or the flexible endoscopic instrument can be assigned to the handpiece, the control element for actuating the pulling element of the at least one Bowden cable is held by the support frame, and the control element can be actuated by means of the actuating element, wherein the handpiece has a guide device for guiding the at least one Bowden cable and/or the cable element, wherein the guide device has a guide element, with at least two opposite guide recesses for passing the cable element through, and a clamping device with at least one clamping element, wherein the guide element is detachably arranged at a distal end of the support frame, the clamping device is detachably arranged on the distal side of the guide element and is non-positively connected to the support frame by means of the at least one clamping element, such that the guide element is held in position between the clamping device and the distal end of the support frame, and the cable element can be guided through the at least two opposite guide recesses in a proximal direction to the control element.

2. Handpiece according to claim 1, wherein the handpiece has the at least one Bowden cable, a second Bowden cable, and/or further Bowden cables, each having a cable element.

3. Handpiece according to claim 1, wherein the support frame has at least one recess, an undercut, and/or at least one projection element in which and/or around which the at least one clamping element engages.

4. Handpiece according to claim 1, wherein the support frame has an aperture, which corresponds to the corresponding guide recess, on either side in the proximal direction for passing the cable element or the cable elements through, or has a smaller width than the guide element.

5. Handpiece according to claim 1, wherein the clamping device has at least two opposite interacting latching hooks, each having a latching lug, which engage around a first projection element on an upper side of the support frame, at least in part.

6. Handpiece according to claim 1, wherein the clamping device has two further opposite interacting latching hooks, each having a latching lug, which engage around a second projection element on an underside of the support frame, at least in part.

7. Handpiece according to claim 1, wherein the clamping device has at least one lateral guide projection on both sides, having a continuous groove in the proximal direction for guiding the at least one Bowden cable or the Bowden cables to the guide recesses of the guide element.

8. Handpiece according to claim 7, wherein the clamping device has two lateral guide projections on both sides, each having a continuous groove in the proximal direction, wherein the two guide projections on either side are arranged one after the other in the proximal direction, and/or the two grooves are arranged in opposite directions to one another.

9. Handpiece according to claim 8, wherein the lateral guide projection arranged on the proximal side has a larger lateral dimension than the lateral guide projection arranged on the distal side, such that the at least one Bowden cable or the Bowden cables expands or expand laterally outwards on both sides in the proximal direction through the two consecutive grooves.

10. Handpiece according to any one of the preceding claims, claim 1, wherein a proximal end of a housing of the at least one Bowden cable or of each Bowden cable rests on a distal side of the guide element around the corresponding guide recess and/or is integrally bonded.

11. Handpiece according to claim 1, wherein the at least two opposite guide recesses each have a guide opening, wherein the guide openings are arranged on both sides in opposite side walls of the guide element in a cross-section transverse to the proximal direction.

12. Handpiece according to claim 11, wherein the at least two opposing guide recesses have, proceeding from the guide opening, a constant recess height, a stepped recess height, and/or a tapering recess height along a recess width.

13. Handpiece according to claim 1, wherein the guide element is designed as a guide plate, wherein a wall thickness of the guide plate is arranged in the proximal direction.

14. Handpiece according to claim 13, wherein the guide plate has an upper recess and a lower recess in a cross-section transverse to the proximal direction, and the support frame has an upper retaining projection corresponding to the upper recess and a lower retaining projection corresponding to the lower recess.

15. Flexible endoscope or flexible endoscopic instrument having a shaft and a bendable shaft portion, wherein the bendable shaft portion is bendable by means of at least one Bowden cable, and having a handpiece arranged at a proximal end of the shaft, wherein the handpiece is a handpiece according to claim 1.
